(19) United States
(12) Plant Patent Application Publication
(10) Pub. No.: US 2013/0276193 P1
(43) Pub. Date: Oct. 17, 2013

(54) Dracaena plant named '2004031C'

(50) ;

(71) Applicant: Ruud Scheffers, Honselersdijk (NL)

(72) Inventor: Ruud Scheffers, Honselersdijk (NL)

(21) Appl. No.: 13/506,403

(22) Filed: Apr. 16, 2012

(51) Int. Cl. A01H 5/00

(52) U.S. Cl. PLT/383

(57) ABSTRACT

A new and distinct Dracaena cultivar named ‘2004031 C’ is disclosed, characterized by very small habit as well as short leaves and short internodes. The new variety is a Dracaena, typically produced as an ornamental plant.

### LATIN NAME OF THE GENUS AND SPECIES

Dracaena fragrans

### VARIETY DENOMINATION

‘2004031C’

### BACKGROUND OF THE INVENTION

The new cultivar is the product of chance discovery. The new variety originated as a naturally occurring mutation of the unpatented Dracaena fragrans ‘Surprise’.

The new variety was discovered by the inventor, Ruud A. M. Scheffers, a citizen of the Netherlands, in August of 2002 in a commercial greenhouse belonging to the inventor in Honselersdijk, The Netherlands. After identifying the new variety as a potentially interesting selection, the inventor continued confidential testing and propagation of ‘2004031C’, assessing stability of the unique characteristics of this variety.

Asexual reproduction of the new cultivar ‘2004031C’ was first performed at the commercial greenhouse of the inventor in Honselersdijk, The Netherlands by vegetative cuttings in August of 2002. Access to all plants was restricted, as plants were kept in a locked greenhouse not open to the public. Through subsequent propagation by vegetative cuttings, multiple generations have been reproduced, which have shown that the unique features cif this cultivar are stable and reproduced true to type. At the time, of filing this application, no plant material of the new variety has been made commercially available.

### SUMMARY OF THE INVENTION

The cultivar ‘2004031C’ has not been observed under all possible environmental conditions. The phenotype may vary somewhat with variations in environment such as temperature, day length, and light intensity, without, however, any variance in genotype.

The following traits have been repeatedly observed and are determined to be the unique characteristics of ‘2004031C’. These characteristics in combination distinguish ‘2004031 C’ as a new and distinct Dracaena cultivar:

1. Very compact plant.

2. Short leaves.

3. Short internodes.

### PARENT COMPARISON

Plants of the new cultivar ‘2004031C’ are similar to the parent, Dracaena fragrans ‘Surprise’ in most horticultural characteristics. The new variety, however, is two to three times smaller at maturity, compared to the parent. Additionally, ‘2004031C’ produces erect leaves compared to the more hanging nature of the parent plant's foliage, as well as solid green leaves, compared to the multi colored foliage of the parent plant.

### COMMERCIAL COMPARISON

‘2004031C’ is similar in most horticultural characteristics to the unpatented variety Dracaena fragrans ‘Janet Craig Compacta’. The two varieties differ in mature size with ‘2004031C’ reaching a mature size two to three times smaller compared to ‘Janet Craig Compacta’.

### BRIEF DESCRIPTION OF THE PHOTOGRAPH

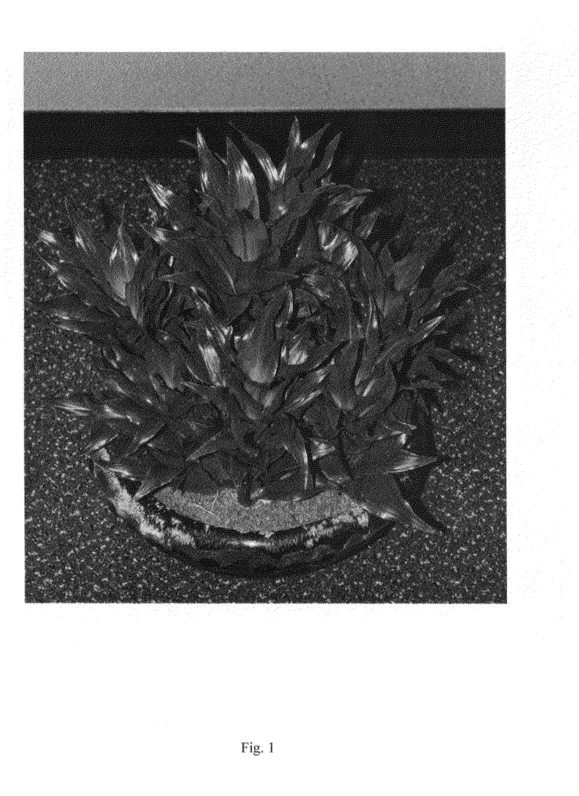

The accompanying photograph in FIG. 1 illustrates in full color a typical plant of ‘2004031C’ grown in a commercial greenhouse in Honselersdijk, The Netherlands. This plant is approximately 5 months old, shown planted in a 15 cm. container. The photograph was taken using conventional techniques and although colors may appear different from actual colors due to light reflectance it is as accurate as possible by conventional photographic techniques.

### DETAILED BOTANICAL DESCRIPTION

In the following description, color references are made to The Royal Horticultural Society Colour Chart 2001, except where general terms of ordinary dictionary significance are used. The following observations and measurements describe ‘2004031C’ plants grown in a greenhouse in Honselersdijk, The Netherlands. A steady temperature of approximately 20° C. at night and 20° C. during the day was maintained. No artificial light, photoperiodic treatments were given to the plants. Measurements and numerical values represent averages of typical plant types.
Botanical classification: Dracaena fragrans ‘2004031C ’
Propagation:
   Time to initiate rooting.—About 4 weeks at approximately 20° C. 6 weeks for full rooting.
   Root description.—Moderately thick, moderately fibrous, not fleshy, young roots Yellow-White 158D, older roots Greyed-Orange 163D.
Plant:
   Age of plant described.—5 months.
   Pot size of plant described.—15 cm. pot.
   Growth habit.—Broad upright.
   Height.—Approximately 17.9 cm.
   Plant spread.—Approximately 11.6 cm.
   Growth rate.—Slow, approximately 3 cm per month.
   Branching characteristics.—Not free branching, growing with one main stem from the base, will only develop lateral branches after pinching/pruning.
   Diameter of stem.—Approximately 0.8 cm. Stem not visible but fully covered by leaves, individual stem characteristics outside of diameter are not measurable.
   Internode length.—Average 0.2 cm.
   Texture of stem.—stem not visible but fully covered by leaves.
   Color of stem.—stem not visible but fully covered by leaves.
   Stem strength.—Very strong.
   Number of leaves per stem.—Average 60.
Foliage:
   Leaf.—Arrangement: Alternate (spirally) Average Length: Approximately 7.7 cm. Average Width: Approximately 2.2 cm. Shape of blade: Lorate Aspect: Average angle at base: 40°, tips curved to near horizontal (90°). 0°=vertical. Apex: Narrow acuminate, not sharp, twisted Base: Broad cuneate Attachment: Sheathing Margin: Entire, slightly undulate Texture of top surface: Smooth, very glossy Texture of bottom surface: Smooth, moderately glossy Color: Young foliage upper side: Near RHS Yellow-Green N144A, tip darker; Green N137A Young foliage under side: Near RHS Green 143C, tip darker; Yellow-Green 146B Mature foliage upper side: Near RHS Green N137A Mature foliage under side: Near RHS Yellow-Green 146A Venation: Type: Parallel Color: Upperside: As leaf blade Underside: As leaf blade.
   Sheath.—Average Length: Approximately 0.8 cm. Average Width: Approximately 0.3 cm. Color: Near RHS Green-White 157D, margins near 157B Texture: Smooth, glossy.
Other characteristics:
   Flower production.—Not observed.
   Disease resistance.—Neither resistance nor susceptibility to normal diseases and pests of Dracaena has been observed.
   Drought tolerance and temperature tolerance.—Moderately high drought resistance, good heat resistance.
   Fruit/seed production.—Not observed.

What is claimed is:

1. A new and distinct cultivar of Dracaena plant named ‘2004031C’ as herein illustrated and described.
